(19) United States
(12) Patent Application Publication
(10) Pub. No.: US 2026/0308088 A1
(43) Pub. Date: Oct. 8, 2026

(54) SELECTIVE HYDRODECHLORINATION OF LIQUID AND GAS STREAMS FROM PLASTICS PYROLYSIS PROCESSES

(71) Applicant: BASF CORPORATION, Florham Park, NJ (US)

(72) Inventors: Artem D. Vityuk, Iselin, NJ (US); Dana Rehms Mooney, Vidalia, LA (US); Armin Lange de Oliveira, Heidelberg (DE); Gisela Hieber, Ludwigshafen (DE); Bernard Reesink, Meern (NL)

(73) Assignee: BASF CORPORATION, Florham Park, NJ (US)

(21) Appl. No.: 19/483,092

(22) PCT Filed: May 19, 2024
(86) PCT No.: PCT/US2024/030133

§ 371 Date: Nov. 10, 2025

Related U.S. Application Data
(60) Provisional application No. 63/467,884, filed on May 19, 2023.

(51) Int. Cl. C07C 7/148; B01J 20/02; B01J 20/04; B01J 20/08; B01J 20/28; B01J 20/34; B01J 21/04; B01J 23/755

(52) U.S. Cl. CPC C07C 7/14841; B01J 20/0244; B01J 20/0277; B01J 20/041; B01J 20/043; B01J 20/08; B01J 20/28011; B01J 20/28016; B01J 20/28061; B01J 20/28071; B01J 20/28073; B01J 20/28076; B01J 20/3433; B01J 20/3458; B01J 21/04; B01J 23/755

(57) ABSTRACT

Described herein are processes and materials for selective hydrodechlorination of liquid and gas streams.

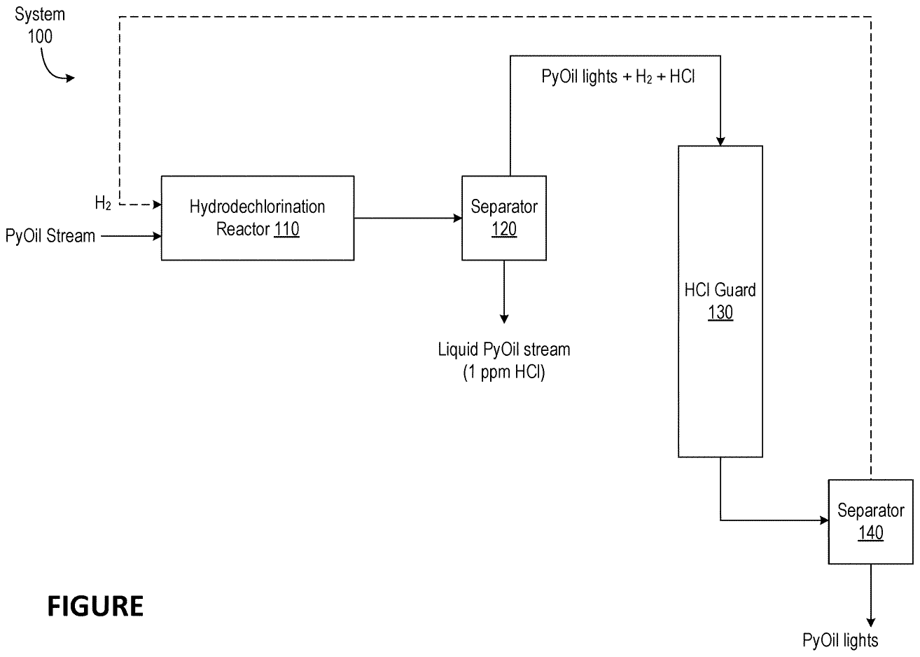

### CROSS-REFERENCE TO RELATED APPLICATIONS

This application claims the benefit of priority of U.S. Provisional Patent Application No. 63/467,884, filed May 19, 2023, the disclosure of which is hereby incorporated by reference herein in its entirety.

### BACKGROUND

Recycling of mixed waste plastics using chemical/monomer recycling is an area of major importance. While multiple chemical recycling methods are being explored, pyrolysis remains the most technically feasible for integration into the steam cracker value chain. Plastics pyrolysis processes that have been commercialized generally prefer to use polyolefins as the feed plastics. However, pure polyolefin waste is rarely accessible in quantities relevant for full industrial scale, and/or expensive presorting needs to be implemented to reject other types of plastics (i.e., polystyrene, polyamide, polyethylene terephthalate, polyvinyl chloride, etc.). It has been shown for small-mid size pyrolysis plants that full exclusion of halogen, nitrogen, and oxygen containing polymers from feed plastics is not currently possible.

Most plastics pyrolysis plants produce liquids with average amounts of the following components: 50-400 ppmw chlorides, 1000-4000 ppmw nitrogen, 2000-20000 ppmw oxygen, and 100-1000 ppmw sulfur. These levels are too high for such liquids to be used as feeds into steam crackers without blending with conventional naphtha. Upgrading of pyrolysis liquids to remove impurities is important to ensure consistency with steam cracker naphtha specifications.

The only commercial solution accessible at scale that allows reduction of these impurities to 1-5 ppmw levels making these liquids drop-in substitute into naphtha crackers is hydroprocessing. Hydroprocessing has been standardized in refining and does not require major process tune ups if to be used for plastics pyrolysis liquids. The only major exception is chlorides/halogens. Hydroprocessing catalysts perform hydrogenation of —N, —O, —S, and —Cl compounds which results in production of respective NH₃, H₂O, H₂S, and HCl. However, there are issues with higher levels of chlorides in such units (i.e. above 10 ppmw), including: (1) formation of NH₄Cl by reaction of NH₃ and HCl, which deposits and clogs equipment at temps below 100° C.; (2) catalysts deactivation; (3) the need for expensive steel. Therefore, there is a need for alternative and/or improved materials and processes.

### SUMMARY

The following summary presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

Disclosed and described herein are catalysts and adsorbents, methods of their preparation, and methods of their use in dechlorination processes.

In one aspect of the present disclosure, a method of removing chlorides from a plastics pyrolysis stream comprises selective hydrodechlorination of PyOil feed using a catalyst that enables removal of chlorides in the form of hydrogen chloride, followed by adsorption of resulting hydrogen chloride with a high performance guard.

In at least one embodiment, selective hydrodechlorination of PyOil is conducted under hydrogen partial pressure in a range of 5-50 barg and at temperature range of 150-350° C.

In at least one embodiment, selective hydrodechlorination of PyOil is conducted under hydrogen partial pressure in a range of 5-40 barg and at temperature range of 200-350° C.

In at least one embodiment, selective hydrodechlorination of PyOil is conducted under hydrogen partial pressure in a range of 5-30 barg and at a temperature range of 200-300° C.

In at least one embodiment, selective hydrodechlorination of PyOil is conducted under hydrogen partial pressure in a range of 5-25 barg and at a temperature range of 250-300° C.

In at least one embodiment, selective hydrodechlorination of PyOil is conducted under hydrogen partial pressure in a range of 10-15 barg and at a temperature of about 300° C.

In at least one embodiment, selective hydrodechlorination of PyOil results in formation of hydrogen chloride in amounts approximately proportional on mole basis to amount of chlorides in the PyOil feed.

In at least one embodiment, the resulting hydrogen chloride is dissolved in PyOil and could be removed using different methods, including but not limited to adsorption, more preferably chemical sorption where HCl reacts with a component of the adsorbent forming a salt.

In at least one embodiment where HCl is removed from PyOil using chemical sorption, a high performance HCl guard is used.

In at least one embodiment, a high performance HCl guard is preferably based on mixed metal oxide formulation, alkali promoted alumina formulation, molecular sieve formulation or combination of these formulations.

In at least one embodiment, a high performance HCl guard is based on mixed metal oxide formulation comprising ZnO, CaO, and alumina.

In at least one embodiment, a high performance HCl guard is based on mixed metal oxide formulation comprising ZnCO₃, CaCO₃, and alumina.

In at least one embodiment, a high performance HCl guard functions at a temperature range of 50-550° C. and at a pressure range of 1-50 barg.

In at least one embodiment, a high performance HCl guard functions at a temperature range of 150-450° C. and at pressure range of 1-40 barg.

In at least one embodiment, a high performance HCl guard functions at temperature range of 200-350° C. and pressure range of 1-30 barg.

In at least one embodiment, a high performance HCl guard functions at temperature range of 250-350° C. and a pressure range of 1-30 barg.

In at least one embodiment, a high performance HCl guard functions at temperature range of 300-350° C. and a pressure range of 1-30 barg.

In at least one embodiment, a high performance HCl guard vessel is installed downstream of a hydrodechlorination vessel.

In at least one embodiment, a selective hydrodechloriantion catalyst and high performance HCl guard are combined in a same vessel.

### BRIEF DESCRIPTION OF THE DRAWING

The disclosure described herein is illustrated by way of example and not by way of limitation in the accompanying drawings.

The FIGURE shows an exemplary system removing chlorides from a plastics pyrolysis stream, in accordance with at least one embodiment.

### DETAILED DESCRIPTION

Embodiments of the present disclosure relate to processes and compositions for the efficient removal of chlorides from plastics pyrolysis streams (which may be liquid or gas streams). Certain embodiments are directed to a plastics pyrolysis oil (PyOil) dechlorination process that comprises selective hydrodechlorination where suitable catalyst is used, followed by HCl adsorption on a high performance guard. These two steps could be combined in a same vessel or performed in different vessels.

Selective hydrodechlorination is a process where organic chlorides in the PyOil are converted to HCl, with minimal hydrogenation of nitrogen species. Limiting hydrogenation of nitrogen species in the PyOil at conditions optimal for hydrogenation of chlorides is crucial. Hydrogenation of nitrogen species normally results in formation of NH₃ and if not limited by selective catalyst, resulting NH₃ could recombine with HCl to form NH₄Cl, a salt that tends to deposit on equipment as solid at temperatures below about 100° C. It is believed that full restriction of nitrogen species hydrogenation is difficult due to high level of —N in waste plastics PyOils and diversity of —N structures in PyOils. The embodiments of the present disclosure advantageously limit nitrogen conversion to about 5-10% while maintaining chlorides conversion at 50-100%, which is sufficient to warrant commercial suitability of the dechlorination process.

In at least one embodiment, selective hydrodechlorination is performed using catalyst formulation determined to be suitable for this purpose. It was found that some conventional hydrotreating catalysts show high performance in selective hydrodechlorination of PyOils if suitable conditions are chosen, i.e., temperature, partial pressure of hydrogen, and LHSV. Typical compositions of hydrotreating catalysts are CoMo, NiMo formulations known to those of ordinary skill in the art. A wide range of CoMo, NiMo, and Ni based catalysts are suitable for hydrodechlorination of PyOils. These catalysts could be used in oxide or sulfide forms. Preferably, such catalysts are in sulfide form where known sulfidation procedures are used to perform sulfidation of the catalyst.

In at least one embodiment, the adsorbent comprises nickel impregnated onto aluminum oxide. In at least one embodiment, the catalytic metal (e.g., nickel) is present from about 10 wt. % to about 30 wt. % based on the total weight of the adsorbent. In at least one embodiment, the catalytic metal may be present at about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, about 26 wt. %, about 27 wt. %, about 28 wt. %, about 29 wt. %, about 30 wt. %, or in any range defined by and inclusive of these points (e.g., from about 12 wt. % to about 17 wt. %).

Hydrodechlorination of PyOil results in formation of HCl which is removed from the stream in certain embodiments using HCl adsorbents, such as one or more high performance HCl guards. In at least one embodiment, suitable HCl adsorbents can include, but are not limited to, mixed metal oxide adsorbents, alkali promoted alumina adsorbents, molecular sieve adsorbents, or combinations thereof. In certain embodiments that utilize HCl guards, such formulations advantageously feature saturation HCl uptake of about 20 wt. % to about 30 wt. %, more preferably of about 25 wt. % to about 35 wt. %. Mixed metal oxide, mixed metal salt, or a combination thereof is preferred. Typical compositions of HCl adsorbents comprise: ZnO, CaO and binding agent; ZnCO₃, CaCO₃ and binding agent (aluminum oxide and or silicon oxide); or a combination thereof. Exemplary HCl guard formulations for use in various embodiments are described in Table 1.

TABLE 1
Exemplary high performance HCl guard formulations

| | | Formula- | Formula- | Formula- | Formula- |
|---|---|---|---|---|---|
| | Units | tion 1 | tion 2 | tion 3 | tion 4 |
| Shape | | Sphere | Sphere | Extrudate | Extrudate |
| | | 3.2 mm | 3.2 mm | 3.2 mm | 3.2 mm |
| LOI (250- | % | 6.5 | 5 | — | — |
| 1100° C.) | | | | | |
| Surface area | m²/g | 250 | 220 | — | — |
| Bulk density | kg/m³ | 675 | 720 | 700 | 750 |
| Crush strength | N | 50 | 80.0-95.0 | 128 | 115 |
| Water pore | mL/g | 0.6 | 0.4 | — | — |
| volume | | | | | |
| Macropore | mL/g | 0.3 | — | — | — |
| volume | | | | | |
| Composition | | | | | |
| Na₂O | wt. % | 5.5 | 8.0-9.0 | none | trace |
| AlOx | wt. % | balance | balance | none | trace |
| ZnO | wt. % | none | none | none | 24.0-25.0 |
| CaO | wt. % | none | none | none | 50.0-52.0 |
| SiO₂ | wt. % | trace | trace | balance | balance |
| CaCO₃ | wt. % | none | none | 40.0-45.0 | none |
| ZnCO₃ | wt. % | none | none | 40.0-45.0 | none |

The adsorbents described may utilize a porous support and one or more active metal components supported thereon. Exemplary supports include metal oxides, metalloid oxides, activated carbons, and molecular sieves. For example, the support may include titanium oxide, ceria, alumina, silica, zirconia, magnesium oxide, zeolites, or combinations thereof. In at least one embodiment, supports include silica. In at least one embodiment, the support may include high surface area metal oxides. In at least one embodiment, the support may comprise aluminum oxide. In at least one embodiment, the support may comprise a mixture of titanium dioxide and aluminum oxide. Metal oxide mixtures, for example a mixture of titanium dioxide and aluminum oxide, may contain metal oxides in a weight/weight ratio of titanium dioxide to aluminum oxide of from any of about 9/1, about 8/1, about 7/1, about 6/1, about 5/1, about 4/1, about 3/1, about 2/1 or about 1/1 to any of about 1/2, about 1/3, about 1/4, about 1/5, about 1/6, about 1/7, about 1/8, or about 1/9.

The adsorbents described herein may be prepared by a variety methods. For instance, a metal may be dispersed onto a support via an incipient-wetness technique. “Impregnated,” in general, means that the materials are “in” pores of the support. In at least one embodiment, the metal is precipitated onto the support. In at least one embodiment, the metal may be reduced after being dispersed or precipitated onto the support.

Surface area, as discussed herein, is determined by the Brunauer-Emmett-Teller (BET) method according to DIN ISO 9277:2003-05 (which is a revised version of DIN 66131), which may be referred to as “BET surface area.” The specific surface area is determined by a multipoint BET measurement in the relative pressure range from 0.05-0.3 p/p₀. In at least one embodiment, the adsorbent has a BET surface area of about 100 m²/g, about 110 m²/g, about 120 m²/g, about 130 m²/g, about 140 m²/g, about 150 m²/g, about 160 m²/g, about 170 m²/g, about 180 m²/g, about 190 m²/g, about 200 m²/g, about 210 m²/g, about 220 m²/g, about 230 m²/g, about 240 m²/g, about 250 m²/g, greater than 250 m²/g, or in any range defined by and inclusive of these points (e.g., from about 150 m²/g to about 200 m²/g).

Pore volume and average pore radius, as discussed herein, are determined by the Barret-Joyner-Halenda (BJH) method. Mercury porosimetry analysis can be used to characterize porosity. Mercury porosimetry applies controlled pressure to a sample immersed in mercury. External pressure is applied for the mercury to penetrate into the voids/pores of the material. The amount of pressure required to intrude into the voids/pores is inversely proportional to the size of the voids/pores. A mercury porosimeter generates volume and pore size distributions from the pressure versus intrusion data generated by the instrument using the Washburn equation:

$$D = \frac{- 4 g cos q}{P} ,$$

where D=diameter, P=pressure, g=surface tension of mercury, q=contact angle. An exemplary method for measuring pore volume may be performed according to ASTM D4284-12 (2017)e1, “Standard Test Method for Determining Pore Volume Distribution of Catalysts and Catalyst Carriers by Mercury Intrusion Porosimetry.” For example, in at least one embodiment, the alumina adsorbent has a total volume of about 0.1 mL/g, about 0.2 mL/g, about 0.3 mL/g, about 0.4 mL/g, about 0.5 mL/g, about 0.6 mL/g, about 0.7 mL/g, about 0.8 mL/g, about 0.9 mL/g, about 1.0 mL/g, about 1.5 mL/g, 2.0 mL/g, about 2.5 mL/g, 3.0 mL/g, about 3.5 mL/g, 4.0 mL/g, about 4.5 mL/g, 5.0 mL/g, about 5.5 mL/g, about 6.0 mL/g, greater than about 6.0 mL/g, or in any range defined by and inclusive of these points (e.g., from about 0.3 mL/g to about 6 mL/g).

In at least one embodiment, the adsorbent has a density of about 0.5 g/mL, about 0.6 g/mL, about 0.7 g/mL, about 0.8 g/mL, about 0.9 g/mL, about 1.0 g/mL, about 1.1 g/mL, about 1.2 g/mL, about 1.3 g/mL, about 1.4 g/mL, about 1.5 g/mL, or in any range defined by and inclusive of these points (e.g., about 0.7 g/mL to about 1.0 g/mL).

The suitable components may be present in the adsorbent compositions in a bulk form, meaning in a continuous form in general not interrupted by other materials. A bulk form may contain substantially no other materials. Accordingly, the adsorbent compositions may be in any suitable final form, for instance tablets, extrudates, pellets, rods, moldings or monoliths, etc., in various shapes and sizes. In at least on embodiment, an adsorbent is in the form of an extruded material, such as extruded particles. In at least one embodiment, the extruded particles are elongated and may have an average extrudate length (i.e., an average largest dimension) of about 1 mm, about 2 mm, about 3 mm, about 4 mm, about 5 mm, about 6 mm, or in any range defined by and inclusive of these points (e.g., from about 3 mm to about 5 mm). In at least one embodiment, the extruded particles exhibit a side crush strength of greater than about 10 N, greater than about 20 N, greater than about 30 N, greater than about 40 N, greater than about 50 N, greater than about 60 N, greater than about 70 N, greater than about 80 N, greater than about 90 N, or greater than about 100 N.

The adsorbents described herein may be suitable for removing chlorides and/or other components from feed streams, such as plastics pyrolysis streams. As used herein, the term “chlorides” refers to chlorine-containing compounds that may include, but not limited to, chloroalkanes, chloroalkenes, chlorooxygenates, chloronaphthenes, and chloroaromatics. Streams suitable for treatment by the adsorbents described herein may include a chlorides content of greater than about 10 ppmw, up to about 500 ppmw (e.g., about 10 ppmw to about 45 ppmw, or about 100 ppmw to about 250 ppmw), or greater.

In at least one embodiment, the adsorbent exhibits a chlorides removal ratio of at least about 90%, at least about 91%, at least about 92%, at least about 93%, at least about 94%, at least about 95%, at least about 96%, at least about 97%, at least about 98%, or at least about 99% when contacted with a pyrolysis liquid stream (e.g., having a chlorides content of greater than about 80 ppmw) at a temperature from about 200° C. to 350° C.

In at least one embodiment, the adsorbent is regenerative (i.e., the adsorbent is capable of being regenerated to restore its activity to or near its activity prior to use). In at least one embodiment, the adsorbent can be regenerated by treating the adsorbent with a regeneration stream. In at least one embodiment, the regeneration stream is at a temperature of about 450° C. to about 600° C. and comprises hydrogen gas. In at least one embodiment, the regeneration stream is at a temperature of about 250° C. to about 350° C. and comprises oxygen gas.

The FIGURE shows an exemplary system 100 including a hydrodechlorination reactor 110 and a downstream HCl guard 130, in accordance with at least one embodiment. In at least one embodiment, the stream after treatment by the hydrodechlorination reactor 110 may be a gas or a liquid. In at least one embodiment, a liquid/gas separation unit (separator 120) separates the stream into a liquid stream having an HCl content of about 1 ppm or less and a gas stream containing PyOil light components, H₂, and HCl. The gas stream is passed through the HCl guard 130 to remove HCl, and to a further separation unit (separator 140) to separate the PyOil light components from the H₂. In at least one embodiment, the H₂ is fed back to the hydrodechlorination reactor 110.

Unless otherwise indicated, all parts and percentages are by weight. Weight percent (wt. %), if not otherwise indicated, is based on an entire composition free of any volatiles, that is, based on dry solids content.

The following exemplary embodiments are now described:
Embodiment 1: A method of removing chlorides from a plastics pyrolysis stream comprising an initial chloride concentration of at least about 10 ppmw, the method comprising: contacting the stream with a catalyst to selectively hydrogenate organic chlorides to form hydrogen chloride, the catalyst comprising a catalytic metal on a support; and subsequently contacting the stream with an adsorbent to remove hydrogen chloride, wherein a final chloride concentration of the treated stream is less than about 10 ppmw.
Embodiment 2: The method of Embodiment 1, wherein the initial chloride concentration is from about 10 ppmw to about 1000 ppmw.
Embodiment 3: The method of any of the preceding Embodiments, wherein the catalytic metal comprises nickel.
Embodiment 4: The method of Embodiment 3, wherein the nickel is present from about 10 wt. % to about 25 wt. %.
Embodiment 5: The method of any of the preceding Embodiments, wherein the support comprises aluminum oxide.
Embodiment 6: The method of any of the preceding Embodiments, wherein the adsorbent comprises a high performance HCl guard.
Embodiment 7: The method of Embodiment 6, wherein the high performance HCl guard comprises a mixed metal oxide formulation, an alkali promoted alumina formulation, a molecular sieve formulation, or a combination thereof.
Embodiment 8: The method of any of the preceding Embodiments, wherein the adsorbent comprises ZnO, CaO, and alumina.
Embodiment 9: The method of any of the preceding Embodiments, wherein the adsorbent comprises ZnCO₃, CaCO₃, and alumina.
Embodiment 10: The method of any of the preceding Embodiments, wherein the adsorbent has a BET surface area of about 150 m²/g to about 300 m²/g.
Embodiment 11: The method of any of the preceding Embodiments, wherein the adsorbent has a total pore volume from about 0.2 mL/g to about 6 mL/g.
Embodiment 12: The method of any of the preceding Embodiments, wherein the adsorbent has a density of about 0.6 kg/m³ to about 1.0 kg/m³.
Embodiment 13: The method of any of the preceding Embodiments, wherein the adsorbent is in the form of extruded particles.
Embodiment 14: The method of Embodiment 13, wherein the extruded particles have an average extrudate length of about 3 mm to about 5 mm.
Embodiment 15: The method of Embodiment 14, wherein the extruded particles exhibit a side crush strength of greater than about 40 N.
Embodiment 16: The method of any of the preceding Embodiments, wherein a chlorides removal ratio is at least about 95% for a temperature of the pyrolysis stream from 200° C. to 350° C.
Embodiment 17: The method of any of the preceding Embodiments, wherein the catalyst is selective for chloride over nitrogen.
Embodiment 18: The method of Embodiment 17, wherein at least about 80 wt. % of organic chlorides in the plastics pyrolysis stream are converted by the catalyst and less than about 1 wt. % of organic nitrogen compounds in the plastics pyrolysis stream are unreacted from contact with the catalyst.
Embodiment 19: The method of any of the preceding Embodiments, wherein the adsorbent is regenerative.
Embodiment 20: The method of Embodiment 19, further comprising: subsequently contacting the adsorbent with a regeneration stream to regenerate the alumina adsorbent.
Embodiment 21: The method of Embodiment 20, wherein the regeneration stream comprises hydrogen gas at a temperature of about 450° C. to about 600° C.
Embodiment 22: The method of Embodiment 20, wherein the regeneration stream comprises oxygen gas at a temperature of about 250° C. to about 350° C.

### ILLUSTRATIVE EXAMPLE(S)

The following example(s) are set forth to assist in understanding the disclosure and should not, of course, be construed as specifically limiting the embodiments described and claimed herein. Such variations of the embodiments, including the substitution of all equivalents now known or later developed, which would be within the purview of those skilled in the art, and changes in formulation or minor changes in experimental design, are to be considered to fall within the scope of the embodiments incorporated herein.

### Example 1: Dechlorination Performance

Standard hydrodechlorination experiments were performed for various samples (described below) in a flow reactor loaded with about 1 mL of a given sample. Hydrodechlorination was performed using a commercial waste PyOil sample containing about 150 ppmw of chlorides, which were organic chlorides. Prior to each experiment, the reactor was purged with dry nitrogen, and samples were dehydrated at 250° C. for one hour at a gas hourly space velocity (GHSV) of 1000 hr⁻¹. After dehydration, dry nitrogen gas was replaced with hydrogen at a total pressure of 50 barg and temperature was increased to 300° C. Once temperature of 300° C. was reached, a liquid PyOil feed was introduced into the reactor at a liquid hourly space velocity (LHSV) of 1 hr⁻¹, total hydrogen pressure was 50 barg. PyOil flow was stabilized through all sample positions of the setup.

Measurements of chlorides and nitrogen in the effluent liquid stream were at performed at 300° C. at varying hydrogen partial pressure, in a range from 50 barg to 2.5 barg, while at constant total pressure of 50 barg. Argon was used as a diluent to sustain total pressure of 50 barg in all experiments. Hydrogen partial pressure reduction was performed in increments: 50 barg, 38 barg, 25 barg, 12.5 barg, 2.5 barg. Each increment was maintained for about 20-24 hours to allow system to equilibrate.

Results presented in Table 2 show that selective hydrodechlorination with respect to chlorides conversion over nitrogen in a PyOil is achieved at different conditions on different catalysts. A catalyst prepared by impregnation and comprising 15 wt % Ni supported on AlOx in oxide form showed reduction of chlorides in PyOil from 150 ppmw to 0-15 ppmw as hydrogen partial pressure was lowered from 50 barg to 2.5 barg at constant temperature of 300° C. In the same experiment, the same catalyst showed extremely poor nitrogen hydrogenation performance, specifically reduction of nitrogen in PyOil from 750 ppmw to 700 ppmw at highest hydrogen partial pressure of 50 barg. There was no nitrogen conversion observed at hydrogen partial pressure of 25 barg and below, at constant temperature of 300 degrees. These results highlight that almost full hydrogenation of chlorides in PyOil is feasible at limited to no hydrogenation of nitrogen on this catalyst at hydrogen partial pressure of 12.5-25 barg and at temperature of 300° C.

A commercial hydrotreating NiMo catalyst in oxide form showed reduction of chlorides in PyOil from 150 ppmw to 0 ppmw as hydrogen partial pressure was lowered from 50 barg to 12.5 barg at constant temperature of 300 degrees. Reduction of chlorides from 150 ppmw to about 9 ppmw was achieved at hydrogen partial pressure of 2.5 barg at 300 degrees. In the same experiment, the same catalyst showed some nitrogen hydrogenation performance, specifically reduction of nitrogen in PyOil from 750 ppmw to 300 ppmw at highest hydrogen partial pressure of 50 barg and to 650 ppmw at lowest hydrogen partial pressure of 2.5 barg at constant temperature of 300 degrees. This result highlights that substantially lower hydrogen partial pressures are needed for this catalyst to enable full chlorides conversion at minimal nitrogen conversions.

TABLE 1
Performance data on selective hydrodechlorination of PyOil.
Experimental conditions: 300° C., total pressure 50
barg, hydrogen partial pressure range 50-2.5 barg, LHSV 1 h⁻¹.

| | Total | Hydrogen | Chloride | Chloride |
|---|---|---|---|---|
| | Pressure | Partial Pressure | in Feed | in Product |
| Sample | (barg) | (barg) | (ppmw) | (ppmw) |
| 15 wt. % Ni | 50 | 50 | 150 | 0 |
| on AlOx | 50 | 38 | 150 | 3.5 |
| | 50 | 25 | 150 | 5 |
| | 50 | 12.5 | 150 | 6 |
| | 50 | 2.5 | 150 | 15 |
| | Total | Hydrogen | Nitrogen | Nitrogen |
| | Pressure | Partial Pressure | in Feed | in Product |
| Sample | (barg) | (barg) | (ppmw) | (ppmw) |
| 15 wt. % Ni | 50 | 50 | 750 | 700 |
| on AlOx | 50 | 38 | 750 | 720 |
| | 50 | 25 | 750 | 750 |
| | 50 | 12.5 | 750 | 750 |
| | 50 | 2.5 | 750 | 750 |
| | Total | Hydrogen | Chloride | Chloride |
| | Pressure | Partial Pressure | in Feed | in Product |
| Sample | (barg) | (barg) | (ppmw) | (ppmw) |
| Commercial | 50 | 50 | 150 | 0 |
| NiMo | 50 | 38 | 150 | 0 |
| | 50 | 25 | 150 | 0 |
| | 50 | 12.5 | 150 | 0 |
| | 50 | 2.5 | 150 | 9 |
| | Total | Hydrogen | Nitrogen | Nitrogen |
| | Pressure | Partial Pressure | in Feed | in Product |
| Sample | (barg) | (barg) | (ppmw) | (ppmw) |
| Commercial | 50 | 50 | 750 | 300 |
| NiMo | 50 | 38 | 750 | 350 |
| | 50 | 25 | 750 | 450 |
| | 50 | 12.5 | 750 | 560 |
| | 50 | 2.5 | 750 | 650 |

It is contemplated that use of a downstream chloride adsorbent could be used in the same reaction vessel or in a separate vessel to adsorb HCl resulting from the hydrodechlorination experiments discussed above. For example, a suitable adsorbent may be a mixed metal oxide formulation (e.g., an adsorbent comprising ZnO, CaO, and alumina; or an adsorbent comprising ZnCO₃, CaCO₃, and alumina), an alkali promoted alumina formulation, a molecular sieve formulation, or a combination thereof.

In the foregoing description, numerous specific details are set forth, such as specific materials, dimensions, processes parameters, etc., to provide a thorough understanding of the embodiments of the present disclosure. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances.

In addition, the use of the terms “a,” “an,” “the,” and similar referents in the context of describing the materials and methods discussed herein (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. Moreover, any permutations of recited values are contemplated as defining the bounds of ranges. For example, a range of 1, 2, or 3 to 4, 5, or 6 is understood to include not just 1 to 4, 1 to 5, 1 to 6, 2 to 4, 2 to 5, etc., but also 1 to 2, 1 to 3, 2 to 3, 4 to 6, etc.

The term “about” used throughout is used to describe and account for small fluctuations that may be introduced via experimental or measurement error (e.g., ±1%). All numeric values are modified by the term “about” whether or not explicitly indicated. Numeric values modified by the term “about” include the specific identified value. For example “about 5.0” includes 5.0.

The term “essentially no” or “substantially no” or “substantially free of” means “not purposefully added” and only trace or inadvertent amounts may be present, for instance ≤5 wt %, ≤4 wt %, ≤3 wt %, ≤2 wt %, ≤1 wt %, ≤0.5 wt % or ≤0.25 wt %, based on the weight of the composition referred to, for example the total adsorbent composition. For example, an adsorbent composition that is substantially free of lead may refer to an adsorbent composition for which lead is below a detectable limit, or its presence has a negligible effect on the performance of the adsorbent.

Reference throughout this specification to “one embodiment,” “certain embodiments,” “one or more embodiments,” “an embodiment,” or “some embodiments” means that a particular feature, structure, material, or characteristic described in connection with the embodiment is included in at least one embodiment of the present disclosure. Thus, the appearances of the phrases such as “in one or more embodiments,” “in certain embodiments,” “in one embodiment,” or “in some embodiments” in various places throughout this specification are not necessarily referring to the same embodiment of the present disclosure. Furthermore, the particular features, structures, materials, or characteristics may be combined in any suitable manner in one or more embodiments.

It is to be understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled. The use of any and all examples, or exemplary language (e.g., “such as”) provided herein, is intended merely to better illuminate the materials and methods and does not pose a limitation on the scope unless otherwise claimed. No language in the specification should be construed as indicating any non-claimed element as essential to the practice of the disclosed materials and methods.

Although the embodiments disclosed herein have been described with reference to particular embodiments it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents, and the above-described embodiments are presented for purposes of illustration and not of limitation.

1. A method of removing chlorides from a plastics pyrolysis stream comprising an initial chloride concentration of at least about 10 ppmw, the method comprising:
contacting the stream with a catalyst to selectively hydrogenate organic chlorides to form hydrogen chloride, the catalyst comprising a catalytic metal on a support; and
subsequently contacting the stream with an adsorbent to remove hydrogen chloride, wherein a final chloride concentration of the treated stream is less than about 10 ppmw.

2. The method of claim 1, wherein the initial chloride concentration is from about 10 ppmw to about 1000 ppmw.

3. The method of claim 1, wherein the catalytic metal comprises nickel.

4. The method of claim 3, wherein the nickel is present from about 10 wt. % to about 25 wt. %.

5. The method of claim 1, wherein the support comprises aluminum oxide.

6. The method of claim 1, wherein the adsorbent comprises a high performance HCl guard.

7. The method of claim 6, wherein the high performance HCl guard comprises a mixed metal oxide formulation, an alkali promoted alumina formulation, a molecular sieve formulation, or a combination thereof.

8. The method of claim 1, wherein the adsorbent comprises ZnO, CaO, and alumina.

9. The method of claim 1, wherein the adsorbent comprises ZnCO₃, CaCO₃, and alumina.

10. The method of claim 1, wherein the adsorbent has a BET surface area of about 150 m²/g to about 300 m²/g.

11. The method of claim 1, wherein the adsorbent has a total pore volume from about 0.2 mL/g to about 6 mL/g.

12. The method of claim 1, wherein the adsorbent has a density of about 0.6 kg/m³ to about 1.0 kg/m³.

13. The method of claim 1, wherein the adsorbent is in the form of extruded particles.

14. The method of claim 13, wherein the extruded particles have an average extrudate length of about 3 mm to about 5 mm.

15. The method of claim 14, wherein the extruded particles exhibit a side crush strength of greater than about 40 N.

16. The method of claim 1, wherein a chlorides removal ratio is at least about 95% for a temperature of the pyrolysis stream from 200° C. to 350° C.

17. The method of claim 1, wherein the catalyst is selective for chloride over nitrogen.

18. The method of claim 17, wherein at least about 80 wt. % of organic chlorides in the plastics pyrolysis stream are converted by the catalyst and less than about 1 wt. % of organic nitrogen compounds in the plastics pyrolysis stream are unreacted from contact with the catalyst.

19. (canceled)

20. The method of claim 1, further comprising:
subsequently contacting the adsorbent with a regeneration stream to regenerate the adsorbent.

21. The method of claim 20, wherein the regeneration stream comprises hydrogen gas at a temperature of about 450° C. to about 600° C., or, wherein the regeneration stream comprises oxygen gas at a temperature of about 250° C. to about 350° C.

22. (canceled)
